(19) United States
(12) Patent Application Publication
(10) Pub. No.: US 2011/0190597 A1
(43) Pub. Date: Aug. 4, 2011

(54) INTERVERTEBRAL DISC ANALYSIS SYSTEM AND METHOD

(76) Inventors: Thierry Marnay, Castelnau-le-Lez (FR); Christophe Geisert, Hofingen (DE); Oliver Frick, Villingen (DE)

(21) Appl. No.: 12/920,810

(22) PCT Filed: Mar. 3, 2008
(86) PCT No.: PCT/EP08/52551

§ 371 Date: Nov. 16, 2010

(51) Int. Cl. A61B 5/00

(52) U.S. Cl. 600/301

(57) ABSTRACT

The invention pertains to an analysis device for measuring physical, chemical and/or biological parameters in an intervertebral disc. It comprises a probe adapted to be inserted in the intervertebral disc; the probe including at its distal end at least three different sensors. The intervertebral disc analysis system and method is useful for measuring physical, chemical and/or biochemical parameters in the intervertebral disc, the data generated by these sensors providing an effigy of the physiological status of the intervertebral disc. The invention further pertains to a method for obtaining physical, chemical and/or biological data of the intervertebral disc, the data being useful for matters in relation to diseases of the intervertebral disc, in particularly for diagnostics and therapies of diseases of the intervertebral disc.

### PRIORITY

The present application is a §371 nationalization of PCT/EP2008/052551, filed Mar. 3, 2008, which is herein incorporated by reference in its entirety, and claims the benefit thereof.

### THE FIELD OF THE INVENTION

The present invention refers to an analysis device and method for the evaluation of the physiological status of the intervertebral disc.

### BACKGROUND

Diseases of the muscular skeletal system particularly of the intervertebral disc are one of the most frequent diseases in Western territory. Lack of exercise, sedentary work and lifestyle, as well as un-physiological movements result in dysfunction of the spine. Thereby, the intervertebral disc is that part of the spine which is mostly affected.

For diagnosis and evaluation and determination of therapy of diseased intervertebral discs the physician can make use of various imaging techniques. A popular method is discography; it is accepted as the intervertebral disc evaluation “procedure-of-choice”. By discography the role of the intervertebral disc in causing the patient's pain is investigated and established. Among experts usefulness of discography is discussed with regard to its indications, value, interpretation of its findings and its safety. Particularly of relevance is the use of fluoroscopic substances which are injected into the intervertebral disc to make lesions and injuries visible. Patients show sometimes allergic reactions towards these fluoroscopic agents. Further, betimes doubtful results of discography are obtained. Discography, like all other imaging techniques, provides the physician with an image of the structures which can be made visible by the specific technique. Physiological conditions are neither shown by discography nor by any other of the imaging techniques applied. Consequently, to properly diagnose a disease of the intervertebral disc as well as for the establishment of precise, tailored and, consequently, effective therapies and treatment plans the knowledge of the physiological status of the intervertebral disc would be of great advantage. Up to date no method or device is known with which the physiological status of the intervertebral disc can be evaluated.

For therapy U.S. Pat. No. 5,433,739 and US 2006/0224223 A1 disclose heating technique whereby a stylet is inserted via a cannula into the intervertebral disc. For monitoring therapy procedure U.S. Pat. No. 5,433,739 discloses use of a temperature sensor, and US 2006/0224223 A1 discloses use of a pressure sensor, and a combination of pressure and temperature sensors, each being integral part of the stylet.

### SUMMARY OF THE INVENTION

Object of the present invention is the provision of a device and method which provides the physician with knowledge of the physiological conditions prevailing in a diseased intervertebral disc.

This object is accomplished by the device of claim 1, the method of claim 11 and the use of claim 13. Preferred embodiments are subject matter of the depending claims.

The analysis device of the invention is for measuring physical, chemical and/or biological parameters in an intervertebral disc. It comprises a probe adapted to be inserted in an intervertebral disc. The probe includes at its distal end at least three different sensors for measuring physical, chemical and/or biochemical parameters in the intervertebral disc. The data generated by these sensors provide an effigy of the physiological status of the intervertebral disc. Preferably there are at least three different sensors selected from the group consisting of a pressure sensor to measure the pressure of the intervertebral disc, a humidity sensor to measure hygrometry of the intervertebral disc, a pH sensor to measure the pH value of the intervertebral disc, a temperature sensor to measure the temperature in the intervertebral disc, a pO2 sensor to measure partial pressure of oxygen, and sensors to measure cytokines's levels, levels of degradation products of proteoglycans, contents of debris and particles accumulated in the intervertebral disc. A presently preferred analysis device includes at least a pressure sensor, a pH sensor, and a humidity sensor. Even more preferred is a combination of pressure sensor, pH sensor, humidity sensor, and temperature sensor.

The invention provides a method for obtaining physical, chemical and/or biological data of the intervertebral disc, the data being useful for matters in relation to diseases of the intervertebral disc, in particularly for diagnostics and therapies of diseases of the intervertebral disc. A probe is inserted into the intervertebral disc, the probe including at least three different sensors for measuring physical, chemical and/or biological parameters in the intervertebral disc, and the data generated by these sensors provide an effigy of the physiological status of the intervertebral disc. Preferably, a cannula is inserted into the intervertebral disc, particularly into the nucleus pulposus. The cannula is adapted to house the probe and the probe is inserted into the intervertebral disc by inserting the probe into the cannula and advancing it to the intervertebral disc, particularly the nucleus pulposus, thereafter the cannula is retracted until the distal end of the probe, including the at least three different sensors, is exposed and measurement of the sensors is commenced.

Finally, use of physical, chemical and/or biological data, generated by at least three different sensors located in the intervertebral disc for evaluation of the physiological status of an intervertebral disc, is disclosed. The sensors are combined in a single probe that is insertable into the intervertebral disc.

The analysis device can be used to retrieve the statistical chemical and physical compounds of intervertebral discs. With the different options available for the correction of the intervertebral disc on the market, these decisions are currently being made of visual determinations only. In the future, analyzing the chemistry of the intervertebral disc prior to diagnosis for correction, will lead to increased success for patients and physicians. This will ensure the recommendation suggested by the physician will equal the successful result expected and desired by patients. Of particular relevance is that the effigy of the physiological status obtained by using the inventive device will lead to better tailored therapy and will reduce the therapeutic burden. It will now be possible to precisely medicate diseases of the intervertebral disc and will help avoiding unnecessary surgery.

The information obtained with the use of the present analysis device will strengthen industry and physicians by allowing worldwide networking, diagnoses, recommendations, research and development for future implants and medications, and ensure success rates for manufacturers of implants and medications, giving them the ability to set their parameters to data received by physicians, and patients.

The probe of the analysis device of the invention is preferably sterilizable or disposable.

Examples of the sensors which can be used are the following. However, other technology is also encompassed by the present invention. The examples following are only for illustration and have been proved useful in practice.

Examples of a temperature sensor are resistance thermometers and thermocouples. Resistance thermometers are constructed in a number of forms and offer great stability, accuracy and repeatability, in some cases they have been proved superior to thermocouples. Resistance thermometers use electrical resistance and require a small power source to operate. The resistance ideally varies linearly with temperature.

An example of a pH sensor is an ion-selective electrode (ISE). ISE is a transducer (sensor) which converts the activity of a specific ion dissolved in a solution into an electrical potential which can be measured by a voltmeter or pH meter. The voltage is theoretically dependent on the logarithm of the ionic activity, according to the Nernst equation. The sensing part of the electrode is usually made as an ion-specific membrane, along with a reference electrode. Ion-selective electrodes are used in biochemical and biophysical research, where measurements of ionic concentration in an aqueous solution are required, usually on a real time basis.

An embodiment of a pressure sensor is a piezoresistive pressure sensor. The sensing material in a piezoresistive pressure sensor is a diaphragm formed on a silicon substrate, which bends with applied pressure. Deformation occurs in the crystal lattice of the diaphragm because of that bending. This deformation effects a change in the band structure of the piezoresistors that are placed on the diaphragm, leading to a change in the resistivity of the material. This change may be an increase or a decrease according to the orientation of the resistors.

Capacitors are examples of humidity sensors. Most capacitors are designed to maintain a fixed physical structure. However, various factors can change the structure of the capacitor; the resulting change in capacitance can be used to sense those factors. The effects of varying the physical and/or electrical characteristics of the dielectric can also be of use. Capacitors with an exposed and porous dielectric can be used to measure humidity in air/fluids.

As briefly discussed, the probe is preferably inserted and housed in a cannula to be inserted into the intervertebral disc. The cannula can be one which is used for discography, also. This has the advantage that the physician can make use of the same cannula for determining the physiological status and for discography. If, for example, the physiological data are not sufficient to allow definite diagnosis the physician can immediately proceed with discography, without need of a second aditus, and vice versa. For that reason, the size of the analysis device is preferably adapted to cannula sizes used in discography. A range of 14 to 17 Gauge, equivalent to 2.03-1.42 mm, is preferred. Miniaturization is aimed at, when technology permits the use of smaller sensors.

An embodiment of the inventive method is as follows: The physician places a needle (14 to 17 Gauge) into the intervertebral disc, particularly its nucleus pulposus, of the patient who is fixed in one position. The present analysis device is inserted into the cannula and advanced until it reaches end of the cannula. In order for the surgeon to uncover sensors of the device for proper measurement, the needle will be retracted a certain distance, e.g. 2 cm, exposing the analysis device to the environment, and measurement of the sensors takes place.

The analysis device is preferably connected to an interface which is in turn connected to a computer. The measurement can be started and stopped by data logging software at anytime by the surgeon. The gathered data will be retained in log file which can be analyzed for example by Windows Excel.

Examples of measurement ranges of a preferred embodiment of the analysis device of the invention are as follows:

pH: 0 to 12

Pressure: 0 to 1333 kPa; burst range: 5332 kPa

Temperature: 0-70° C.

Humidity: 0-100%

Calibration of the sensors is preferred. Sensors can be calibrated before or after measurement. Because sterilization of the probe might considered necessary, calibration of sensors after measurement is preferred.

### BRIEF DESCRIPTION OF THE DRAWINGS

In the following embodiments of the invention will be described. The preferred embodiments are not to be construed as limiting the invention. The attached drawings show:

FIG. 1 an analysis device of the invention in use;

FIG. 2 an embodiment of a probe of the analysis device;

FIG. 3 another embodiment of a probe;

FIG. 4 a further embodiment of a probe;

FIG. 5 a still further embodiment of a probe;

FIG. 6 a still further embodiment of a probe;

FIG. 7 a detail of the analysis device of the invention according to FIG. 1; and

FIG. 8 a still further embodiment of a probe in a sectional view.

### DETAILED DESCRIPTION

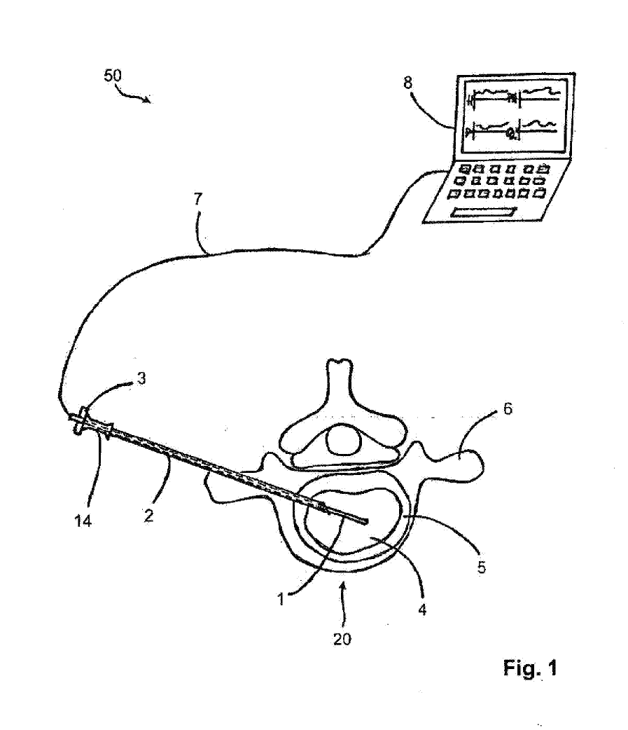

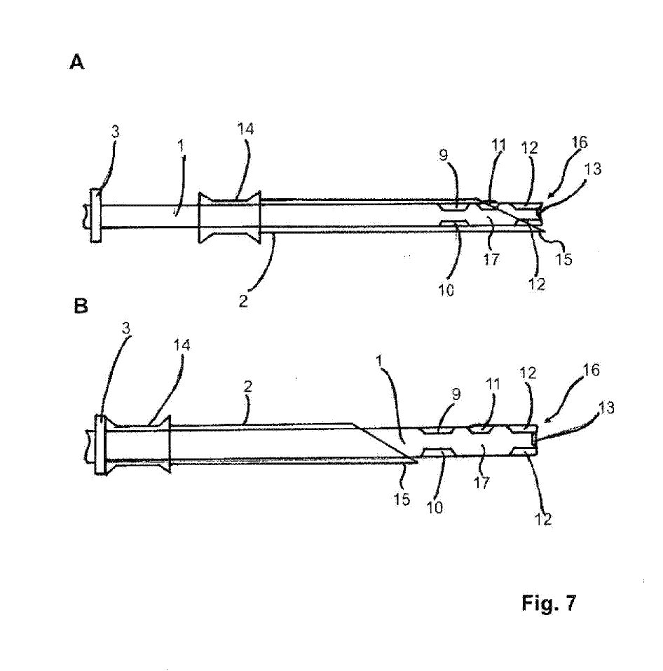

In FIG. 1 a section of a spine is shown schematically. Reference numeral 6 denotes the vertebral body. Reference numeral 20 identifies the intervertebral disc consisting of an outer annulus fibrosus 5, which surrounds the inner nucleus pulposus 4. The analysis device 50 of the embodiment shown in FIG. 1 comprises a probe 1 connected via line 7 with an evaluation unit 8 where data are analyzed, e.g. a computer. The probe 1 is inserted into the intervertebral disc 20 via a cannula 2. Details of a preferred embodiment of cannula 2 and the inserted probe are shown in FIG. 7. As can be seen by reflecting FIGS. 1 and 7 cannula 2 is introduced into the intervertebral disc whereupon probe 1 is advanced through the inner bore of cannula 2 until it reaches the tip 15 of cannula 2. As can be seen from FIG. 7A, after insertion and advance of the probe 1 in cannula 2 the sensors (designated by reference numerals 9 to 13) are still located within the lumen of cannula 2. To expose the sensors to the surrounding intervertebral disc, particularly the nucleus pulposus, cannula 2 is retracted. For this purpose, handle 14 is provided at the distal end of cannula 2, i.e. at the end opposite to the tip 15. Probe 1 carries a distance ring 3. The distance ring 3 is arranged proximally to the sensors which are located at the lateral probe shell 17 near the tip 16 of the probe 1. Distance ring 3 provides a stop position to cannula 2 which bears against distance ring 3 when it is retracted. Consequently, distance ring 3 is positioned on probe 1 in such distance to probe tip 16 that after retraction of the cannula 2 sensors 9 to 13 are exposed to the intervertebral disc in a sufficient manner to perform measurement. Preferably, the cannula is only retracted to such an extent that it still provides a guide and sheath to the probe 1. In other words, after retraction of cannula 2 all sensors 9 to 13 located at the distal end of probe 1 should be exposed to the circumjacent tissue. However, the remaining, proximally located parts of probe 1 should still be housed in the interior of cannula 2. Consequently, depending on location and arrangement of the sensors on probe 1 and the overall length of probe 1, the distance ring 3 is to be spaced accordingly.

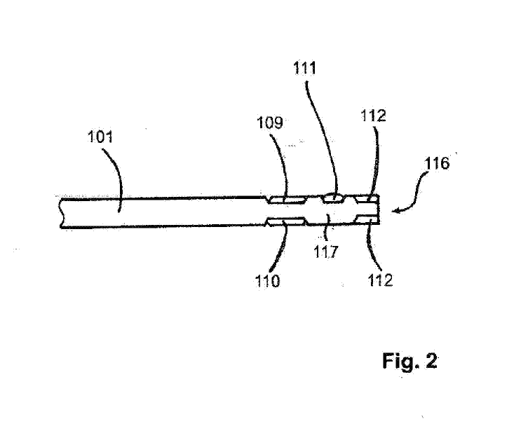

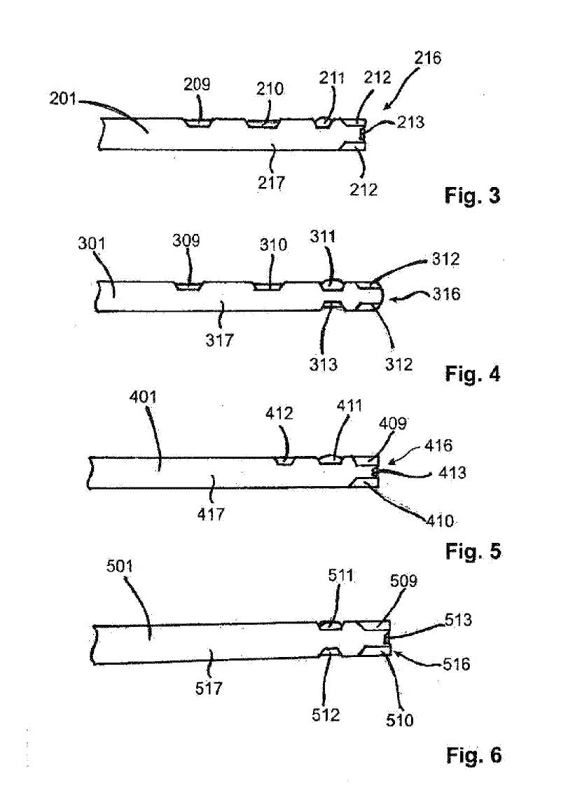

FIGS. 2 to 6, each, show different arrangements of sensors included in analysis device 50. It is to be noted that only a detail is shown, namely the distal parts of the probes, where sensors are arranged. FIG. 2 shows a three-sensor model, whereas FIGS. 3 to 6 each show four-sensor options. As can be seen from all FIGS. 2 to 6 sensor elements are preferably arranged at the distal end of the probe, i.e. the end of the probe which is inserted into the intervertebral disc, opposing that end which is connected via line 7 with the evaluation unit 8.

In FIG. 2 a three-sensor model is depicted. Probe 101 includes pressure sensor 109, humidity sensor 110, and pH sensor 111. Reference electrodes for the pH sensor 111 are also arranged at the probe; they are marked with reference numerals 112. All sensors, as such, are arranged around the distal end of the probe 101. One can also say they are arranged on the lateral probe shell 117. Equivalent to the term “lateral probe shell” can be regarded the terms “mantle” or “surface of the probe 101”. Reference electrodes 112 of the pH sensor 111 are disposed on the tip 116 of the probe 101, whereby the reference electrodes 112 reach into the lateral probe shell 117. Pressure sensor 109 and pH sensor 111 are arranged in line, one after the other, seen from the one end of the probe 101 to the other end, whereas humidity sensor 110 is arranged across from pressure sensor 109 and pH sensor 111.

In the embodiment of FIG. 3—a four-sensor model—pressure sensor 209, humidity sensor 210 and pH sensor 211 are located in a row on the lateral probe shell 217. Further included is a combined temperature and pO2 sensor 213. This combined sensor 213 is placed on the tip 216 of the probe 201. Again, reference electrodes 212 of the pH sensor 211 are included in probe 201.

FIG. 4 shows a further embodiment of a probe 301 where pressure sensor 309, humidity sensor 310 and pH sensor 311 are arranged consecutively, one after the other, with a certain distance to each other at the lateral probe shell 317. The distance between the individual sensors is to be chosen that not any interference between the sensors 309 to 311 occurs. Reference electrodes 312 are again provided at the tip 316 of the probe. The tip 316 of the probe 301 according to the embodiment shown in FIG. 4 is rounded to ensure that probe 301 does not harm the intervertebral disc when advancing and positioning the probe in the intervertebral disc. A combination of temperature and pO2 sensor 313 is located opposite to pH sensor 311.

FIGS. 5 and 6, each show still further embodiments of the inventive probe. At the tip 416 and 516 of the probe 401 and 501 combined sensors 413 and 513 to measure temperature and pO2 level, pressure sensor 409 and 509, and humidity sensor 410 and 510, respectively, are arranged. PH sensor 411 and 511 and its reference electrode 412 and 512 are placed on the lateral probe shell 417 and 517, respectively. In FIG. 5, the reference electrode 412 is arranged parallel to, in line with the pH sensor 411, whereas in FIG. 6 reference electrode 512 is arranged opposite to the pH sensor 511.

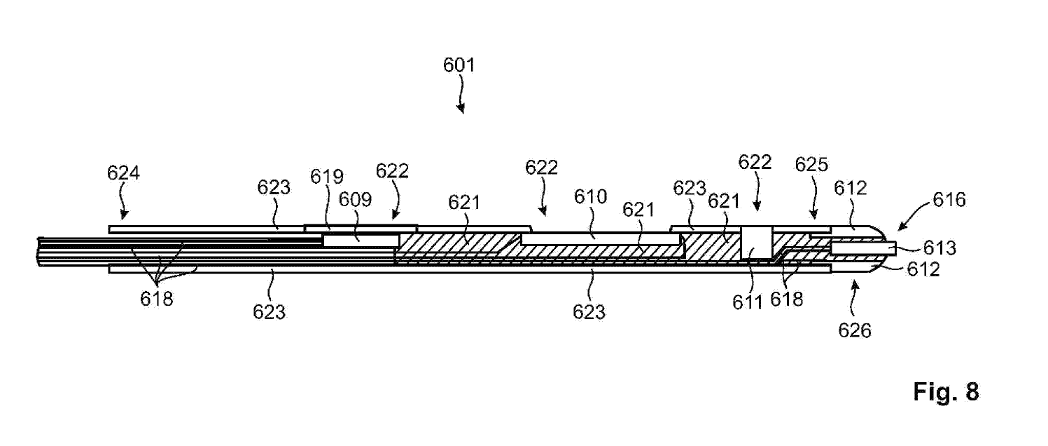

FIG. 8 shows a longitudinal section of an embodiment of probe 601 in a graphical representation. At the tip 616 of the probe 601 sensor 613, combining temperature and pO2 measurement, is located. The combined sensor 613 is flanked by reference electrodes 612 of the pH sensor 611. The tip 616 has rounded edges to present the combined sensor 613 to environment optimally. As can be seen, combined sensor 613 protrudes over the tip 616. PH sensor 611 is arranged close to its reference electrodes 612. By this measure reliability of measurements of the pH sensor is ensured. Humidity sensor 610 is located in an aperture 622 of the probe 601. Pressure sensor 609 is also located in an aperture of the probe. It is covered by a silicone membrane 619. Pressure sensor 609, humidity sensor 610 and pH sensor 611 are spatially separated from each other, but are arranged in a row. Electrical wires 618 of all sensors are bundled in the centre of the probe 601 and are passed towards evaluation unit 8, as shown in FIG. 1.

FIG. 8 also shows a preferred construction of the probe 601. A flexible plastics tube 623 has apertures 622 at those locations where sensors are to be mounted. Mounting of the sensors is performed in that the individual sensors are placed in its assigned apertures 622, carrying electrical wires 618, each. Adhesive 621 (hatched), e.g. epoxy resin or UV curable resin, is filled in for fixation of the sensors 609, 610, 611, 612, 613. As can be seen in FIG. 8, the head 626 of the probe 601 is built up of sensor 613, reference electrodes 612 and adhesive 621. Following thereafter is tube 623, filled with adhesive 621 and carrying further sensors, sensors 611, 610 and 609. Adhesive 621 is only applied as far as sensors reach and as it is necessary for fixation purposes. In the probe 601 of FIG. 8 adhesive 621 reaches from the front end 625 of the probe 601 to the pressure sensor 609, but pressure sensor 609 is not completely embedded in adhesive 621. By this construction, the head 626 and those parts of the probe 601 carrying sensors is solid due to adhesive 621. Thereafter, due to flexibility of the tube 623, the probe 601 is flexible. The tip 616 is particularly firm, this supports advance of the probe 601 in cannula 2. The described construction of probe 601 is of advantage for its insertion into the intervertebral disc 20. The probe 601 can be constructed in any length desired due to the material used, flexible tube 623, which has no fixed length. Apertures 622 for insertion of various sensors are prepared in the near of the front end 625 of tube 623, whereas at its rear end 624 the electrical wires 618 leave the probe 601. Preferably, probe 601 has minimum length of approximately 20 cm, as typically used cannulas for discography and for the purposes of this invention have a length of 20 cm. Even more preferred probe 601 is longer than 20 cm. At its end a plug can be mounted for connecting it to the evaluation unit 8 or any other apparatus.

What is claimed is:

1. Analysis device (50) for measuring physical, chemical and/or biological parameters in an intervertebral disc (20), comprising a probe (1, 101, 201, 301, 401, 501, 601) adapted to be inserted in the intervertebral disc the probe including at its distal end at least three different sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) for measuring physical, chemical and/or biochemical parameters in the intervertebral disc, the data generated by these sensors providing an effigy of the physiological status of the intervertebral disc.

2. Analysis device according to claim 1, wherein the at least three different sensors are selected from the group consisting of pressure sensor (9, 109, 209, 309, 409, 509, 609) to measure the pressure of the intervertebral disc, humidity sensor (10, 110, 210, 310, 410, 510, 610) to measure hygrometry of the intervertebral disc, pH sensor (11, 12, 111, 112, 211, 212, 311, 312, 411, 412, 511, 512, 611, 612) to measure pH of the intervertebral disc, temperature sensor (13, 213, 313, 41, 513, 613) to measure the temperature in the intervertebral disc, pO2 sensor (13, 213, 313, 413, 513, 613) to measure partial pressure of oxygen, sensors to measure cytokines's levels, levels of degradation products of proteoglycans, contents of debris and particles accumulated in the intervertebral disc.

3. Analysis device according to claim 2, wherein the pressure sensor (9, 109, 209, 309, 409, 509, 609) is a piezoresistive pressure sensor, the humidity sensor (10, 110, 210, 310, 410, 510, 610) is a capacitor, the temperature sensor (13, 213, 313, 41, 513, 613) is a resistance thermometer, and the pH sensor (11, 12, 111, 112, 211, 212, 311, 312, 411, 412, 511, 512, 611, 612) is an ion-selective electrode.

4. Analysis device according to claim 2 or 3, wherein the probe (1, 101, 201, 301, 401, 501, 601) includes a pressure sensor (9, 109, 209, 309, 409, 509, 609), a humidity sensor (10, 110, 210, 310, 410, 510, 610) and a pH sensor (11, 12, 111, 112, 211, 212, 311, 312, 411, 412, 511, 512, 611, 612).

5. Analysis device according to any of the preceding claims, wherein the probe (1, 101, 201, 301, 401, 501, 601) fits into a cannula (2), which is insertable into the intervertebral disc (20), and wherein the distal end of the probe is exposable to the surrounding intervertebral disc.

6. Analysis device according to claim 5 wherein the cannula (2) has at its proximal end a handle (14) which interacts with a distance ring (3) acting as an edge guide for the handle and being proximal to the handle, the distance ring being included in the probe (1) and being arranged proximally to the different sensors (9, 10, 11, 12, 1, 109, 110, 111, 112, 209, 210, 211, 212, 213, 309, 310, 311, 312, 313, 409, 410, 411, 412, 413, 509, 510, 511, 512, 513, 609, 610, 611, 612, 613) at the distal end of the probe, the interaction of handle and distance ring being in that the sensors are exposed to the surrounding intervertebral disc if the handle bears against the distance ring.

7. Analysis device according to any of the preceding claims, wherein the different sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) are distributed at the distal end of the probe, the probe tip (16, 116, 216, 316, 416, 516, 616) carrying at least one sensor and the lateral probe shell (17, 117, 217, 317, 417, 517, 617) carrying at least one sensor.

8. Analysis device according to any of the preceding claims having electrical lines (618) placed in the interior of the probe (1, 101, 201, 301, 401, 501, 601), wherein the at least three, particularly four different sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) are arranged in the circumference of the probe, the sensors being spatially separated from each other.

9. Analysis device according to any of claims 2 to 8, wherein one of the at least three sensors is a pH sensor (11, 111, 211, 311, 411, 511, 611) and wherein the reference electrode (12, 112, 212, 312, 412, 512, 612) for the pH sensor is included in the probe (1, 101, 201, 301, 401, 501, 601), particularly in the tip (16, 116, 216, 316, 416, 516, 616) of the probe.

10. Analysis device according to any of the preceding claims, wherein the probe (1, 101, 201, 301, 401, 501, 601) comprises of a lateral probe shell (17, 117, 217, 317, 417, 517, 617) in the form of a tube (623) which is flexible, wherein apertures (622) are provided in the tube (623) for insertion of the at least three sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) and wherein adhesive (621) is filled into the tube for fixation of the sensors and reinforcement of the probe.

11. Method for obtaining physical, chemical and/or biological data of the intervertebral disc (20), the data being useful for matters in relation to diseases of the intervertebral disc, in particularly for diagnostics and therapies of diseases of the intervertebral disc, wherein a probe (1, 101, 201, 301, 401, 501, 601) is inserted into the intervertebral disc, the probe including at least three different sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) for measuring physical, chemical and/or biological parameters in the intervertebral disc, the data generated by these sensors providing an effigy of the physiological status of the intervertebral disc.

12. Method according to claim 11, wherein a cannula (2) is inserted into the intervertebral disc, particularly into the nucleus pulposus (4), the cannula being adapted to house the probe (1, 101, 201, 301, 401, 501, 601) and the probe is inserted into the intervertebral disc by inserting the probe into the cannula and advancing it to the intervertebral disc, particularly the nucleus pulposus, thereafter the cannula is retracted until the distal end of the probe including the at least three different sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) is exposed and measurement of the sensors is commenced.

13. Use of physical, chemical and/or biological data generated by at least three different sensors (9, 10, 11, 12, 13, 109, 110, 111, 112, 209, 210, 211, 212, 213, 309, 310, 311, 312, 313, 409, 410, 411, 412, 413, 509, 510, 511, 512, 513, 609, 610, 611, 612, 613) located in the intervertebral disc (20) for evaluation of the physiological status of an intervertebral disc, wherein the sensors are combined in a single probe (1, 101, 201, 301, 401, 501, 601) that is insertable into the intervertebral disc.
